(19) United States
(12) Patent Application Publication
(10) Pub. No.: US 2014/0024826 A1
(43) Pub. Date: Jan. 23, 2014

(54) SYSTEMS AND METHODS FOR IMPROVING FERMENTATION

(71) Applicants: Neelakantam V. Narendranath, Sioux Falls, SD (US); Steven T. Bly, Yankton, SD (US)

(72) Inventors: Neelakantam V. Narendranath, Sioux Falls, SD (US); Steven T. Bly, Yankton, SD (US)

(21) Appl. No.: 13/980,419

(22) PCT Filed: Jan. 20, 2012
(86) PCT No.: PCT/US12/22065

§ 371 Date: Oct. 9, 2013

Related U.S. Application Data
(60) Provisional application No. 61/435,149, filed on Jan. 21, 2011.

(51) Int. Cl. C07H 1/08

(52) U.S. Cl. CPC C07H 1/08
USPC 536/128; 210/203

(57) ABSTRACT

Systems and methods for increasing fermentation efficiency of a lignocellulosic hydrolysate are disclosed. The system comprises a filter configurable to remove matter having a particle size of larger than about 25 to 100 microns from the liquid component, and at least one nanofilter configurable to remove acids from the liquid component. An apparatus is used to adjust the pH of the nanofiltered liquid component using a calcium hydroxide composition to a pH of about 5.5 to 6.0. The calcium hydroxide composition includes calcium hydroxide alone or in combination with either ammonium hydroxide and/or potassium hydroxide. The biomass comprises lignocellulosic material including at least one of corncob, corn plant husk, corn plant leaves, and corn plant stalks.

### CROSS-REFERENCE TO RELATED APPLICATIONS

This application claims the benefit of U.S. Provisional Application Ser. No. 61/435,149, filed Jan. 21, 2011, and entitled “SYSTEMS AND METHODS FOR IMPROVING FERMENTATION”, the disclosure of which is incorporated herein by reference.

### FIELD

The disclosed aspects relate to systems and methods for improving the fermentation efficiency of lignocellulosic hydrolysates using nano-filtration and a calcium hydroxide composition.

### BACKGROUND

As a preliminary step in a lignocellulosic process, dilute acid pretreatment is an effective means of hydrolyzing a significant portion of structural polysaccharides to monomer sugars and more easily digestible polysaccharide chains. In this process, the feedstock is ground to a suitable size and subjected to a pretreatment process, where the feedstock is exposed to an acid and an elevated temperature. The pretreatment process causes the feedstock to be broken down into a slurry. To substantially separate the pentose containing components of the slurry from the hexose containing components, a process is undertaken that includes separating the liquid component of the slurry, containing a substantial concentration of pentose, from the solid component of the slurry, containing a substantial concentration of hexose. After the slurry separation stage, the pentose liquor may contain impurities, or inhibitors, which may interfere with fermentation. It is well documented that a broad range of compounds are liberated and formed during the acid hydrolysis, and many are toxic to the fermenting microorganism (i.e. fermentation inhibitors) (Klinke et al., 2004; Musatto and Roberto, 2004; Palmqvist and Hahn-Hagerdal, 2000). Known fermentation inhibitors include furan derivatives, furfural and 5-hydroxy-methylfurfural (HMF); aliphatic acids, such as acetic acid, formic acid, and levulinic acid; and phenolic compounds from the breakdown of lignin.

A variety of strategies have been devised to detoxify compounds produced during dilute acid pretreatment or hydrolysis so fermentation may proceed favorably. Some detoxification methods, such as treatment with charcoal or calcium hydroxide (also known as over-liming), have been reported to cause sugar losses, which negatively impacts the overall process yield, already limited by sugar content. One biological method including inoculation with laccases (lignin-degrading enzymes), may be as costly as (or more costly than) the cellulase enzymes needed for the complete digestion of the polysaccharides. Likewise, it has been shown that fermentation of hydrolysates with very large yeast inoculation levels is another effective means for dealing with inhibitors (Chung and Lee, 1984). A large amount of yeast in the inoculation is required due to massive cell death during fermentation. Another mitigation method includes an ion exchange process. While ion exchange may be effective at removing much of the inhibitory compounds found in the pentose liquor, it may be a relatively expensive means for mitigating inhibitors. Another mitigation technique includes nano-filtration. Nano-filtration has been shown to remove acetic acid from pentose liquor, but does little to reduce other inhibitors. It is always desirable to increase the efficiency of inhibitor removal in order to increase fermentation yields.

Currently, the combination of nano-filtration with the addition of calcium hydroxide is not performed because it is known that calcium hydroxide fouls membranes, and can lead to scaling on evaporator and distillation equipment. A system capable of combining inhibitor removal techniques may reduce inhibitor levels in the pentose liquor in a cost effective manner. With fewer inhibitors, fermentation of the liquor occurs more efficiently.

### SUMMARY

The disclosed aspects relate to systems and methods for increasing the efficiency of fermentation of a hydrolysate. A system includes treating a liquid component separated from biomass to yield a treated liquid component comprising sugars available to be fermented into a fermentation product. The biomass comprises lignocellulosic material, which can comprise at least one of corn cob, corn plant husk, corn plant leaves, and corn plant stalks.

The system comprises a filter configured to remove matter having a particle size of larger than about 0.1 to 20 microns from the liquid component. In some embodiments, the filter has a pore size of 0.1 to 20 micrometers.

The system also includes at least one nanofilter configured to remove acids and concentrate xylose from the filtered liquid component. In some embodiments, at least one nanofilter includes a first nanofiltration stage and a second nanofiltration stage. The second nanofiltration stage may comprise a membrane with pores that allow water molecules and acid ions to pass as permeate and retain sugar molecules as retentate. The second nanofiltration stage may also be configured for diafiltration. Diafiltration can include adding water to the liquid component in a ratio of 0:1 to 1.3:1. The first nanofiltration stage has a permeate flux rate of 1.5 to 35 L/m²/h.

The system also includes an apparatus configured to adjust the pH of the nanofiltered liquid component. In some embodiments, the apparatus adjusts pH of the nanofiltered liquid component to about 5.5 to 6.0 using calcium hydroxide. In other embodiments, the apparatus adjusts pH of the nanofiltered liquid component to about 5.5 to 6.0 using a combination of calcium hydroxide and at least one of ammonium hydroxide and potassium hydroxide. In some embodiments, the apparatus adjusts pH of the nanofiltered liquid component to about 4.0 using calcium hydroxide and then adjusts the pH to about 5.5 to 6.0 with at least one of ammonium hydroxide and potassium hydroxide.

Another aspect relates to a method for treating a liquid component separated from biomass to yield a treated liquid component comprising sugars available to be fermented into a fermentation product. The method comprises removing matter having a particle size of larger than about 25 microns from the liquid component. The method also comprises removing acids and concentrate xylose in the liquid component and adjusting a pH of the liquid component using a calcium hydroxide composition.

The biomass can comprise lignocellulosic material. The lignocellulosic material can comprises at least one of corn cob, corn plant husk, corn plant leaves, and corn plant stalks.

In accordance with some aspects, removing the matter comprises using a filter with a pore size of 0.1 to 20 micrometers. According to some aspects, removing comprises using at least one nanofilter including a first nanofiltration stage and a second nanofiltration stage.

In accordance with some aspects, the first nanofiltration stage has a permeate flux rate of 1.5 to 35 L/m2/h. In an aspect, the second nanofiltration stage comprises a membrane with pores that allow water molecules and acid ions to pass as permeate and retain sugar molecules as retentate. Further to this aspect, the liquid component comprises the retentate. According to some aspects, the second nanofiltration stage is configured for diafiltration. Further to this aspect, the diafiltration comprises adding water to the liquid component in a ratio of 0:1 to 1.3:1.

In some aspects, adjusting the pH of the liquid component comprises adjusting the pH to about 5.5 to 6.0 using calcium hydroxide. According to some aspects, adjusting the pH of the liquid component comprises adjusting the pH to about 5.5 to 6.0 using a combination of calcium hydroxide and at least one of ammonium hydroxide and potassium hydroxide.

### DESCRIPTION OF THE DRAWINGS

In order that the disclosed aspects may be more clearly ascertained, some embodiments will now be described, by way of example, with reference to the accompanying drawings, in which:

FIG. 1A is a perspective view of a biorefinery comprising an ethanol production facility, in accordance with some embodiments.

FIG. 1B is a perspective view of a biorefinery comprising an ethanol production facility, in accordance with some embodiments.

FIG. 2 is a system for the preparation of biomass delivered to a biorefinery, in accordance with some embodiments.

FIGS. 3A and 3B are alternative embodiments of a schematic diagram of the cellulosic ethanol production facility in accordance with some embodiments.

FIG. 4A is a process flow diagram illustrating the pretreatment process, in accordance with some embodiments.

FIG. 4B is a schematic perspective view of the pretreatment process, in accordance with some embodiments.

FIG. 5A is a first schematic view of an inhibitor mitigation system, in accordance with some embodiments.

FIG. 5B is a second schematic view of the inhibitor mitigation system, in accordance with some embodiments.

FIG. 6 is a logical block diagram of the inhibitor mitigation system, in accordance with some embodiments.

FIG. 7 is a process flow diagram of the inhibitor mitigation system, in accordance with some embodiments.

FIG. 8A to 8C provide operating conditions for the nano-filtration, in accordance with some embodiments.

FIG. 9A is a schematic diagram of a process flow for an experimental process, in accordance with some embodiments.

FIG. 9B is a schematic diagram of the principle of concentration and diafiltration.

FIGS. 10 through 17 are graphs of the results of treatment of the liquid stream according to an exemplary embodiment.

FIG. 18 is an example graph illustrating changes in ethanol yields in relation to fermentation time for samples of varying initial xylose concentrations and pH adjustments, in accordance with some embodiments.

FIG. 19 is an example graph illustrating changes in residual xylose concentrations in relation to fermentation time for samples of varying initial xylose concentrations and pH adjustments, in accordance with some embodiments.

FIG. 20 is an example graph illustrating changes in ethanol yield concentrations in relation to fermentation time for samples adjusted for pH using lime or potassium hydroxide, in accordance with some embodiments.

FIG. 21 is an example graph illustrating changes in residual xylose concentrations in relation to fermentation time for samples adjusted for pH using lime or potassium hydroxide, in accordance with some embodiments.

FIG. 22 is an example graph illustrating changes in ethanol yield concentrations in relation to fermentation time for samples adjusted for pH using lime or ammonium hydroxide, in accordance with some embodiments.

FIG. 23 is an example graph illustrating changes in ethanol yield concentrations in relation to fermentation time for samples adjusted for pH using lime or a combination of lime with ammonium hydroxide, in accordance with some embodiments.

FIG. 24 is an example graph illustrating changes in residual xylose concentrations in relation to fermentation time for samples adjusted for pH using lime or a combination of lime with ammonium hydroxide, in accordance with some embodiments.

TABLES 1A and 1B list the composition of biomass comprising lignocellulosic plant material from the corn plant according to exemplary and representative embodiments.

TABLES 2A and 2B list the composition of the liquid component of pre-treated biomass according to exemplary and representative embodiments.

TABLES 3A and 3B list the composition of the solids component of pre-treated biomass according to exemplary and representative embodiments.

TABLE 4A is an experimental design for an exemplary embodiment.

TABLE 4B lists the composition of samples from an exemplary embodiment.

TABLE 5A is an experimental design for an exemplary embodiment.

TABLE 5B lists the composition of samples from an exemplary embodiment.

### DESCRIPTION OF THE EMBODIMENTS

The various aspects will now be described in detail with reference to several embodiments thereof as illustrated in the accompanying drawings. In the following description, numerous specific details are set forth in order to provide a thorough understanding of embodiments of the one or more aspects. It will be apparent, however, to one skilled in the art, that embodiments may be practiced without some or all of these specific details. In other instances, well known process steps and/or structures have not been described in detail in order to not unnecessarily obscure the disclosed aspects. The features and advantages of embodiments may be better understood with reference to the drawings and discussions that follow.

The disclosed aspects relate to systems and methods for improving fermentation though the mitigation of fermentation inhibitors in the liquid portion of lignocellulosic hydrolysate using a combination of nanofiltration and the addition of lime (calcium hydroxide). Aspects provide for decreasing inhibitors resulting from lignocellulosic hydrolysates. Various aspects also provide an improvement in the reduction of fermentation inhibitors, such as furfural. The disclosed systems and methods provide an effective method of improving fermentation.

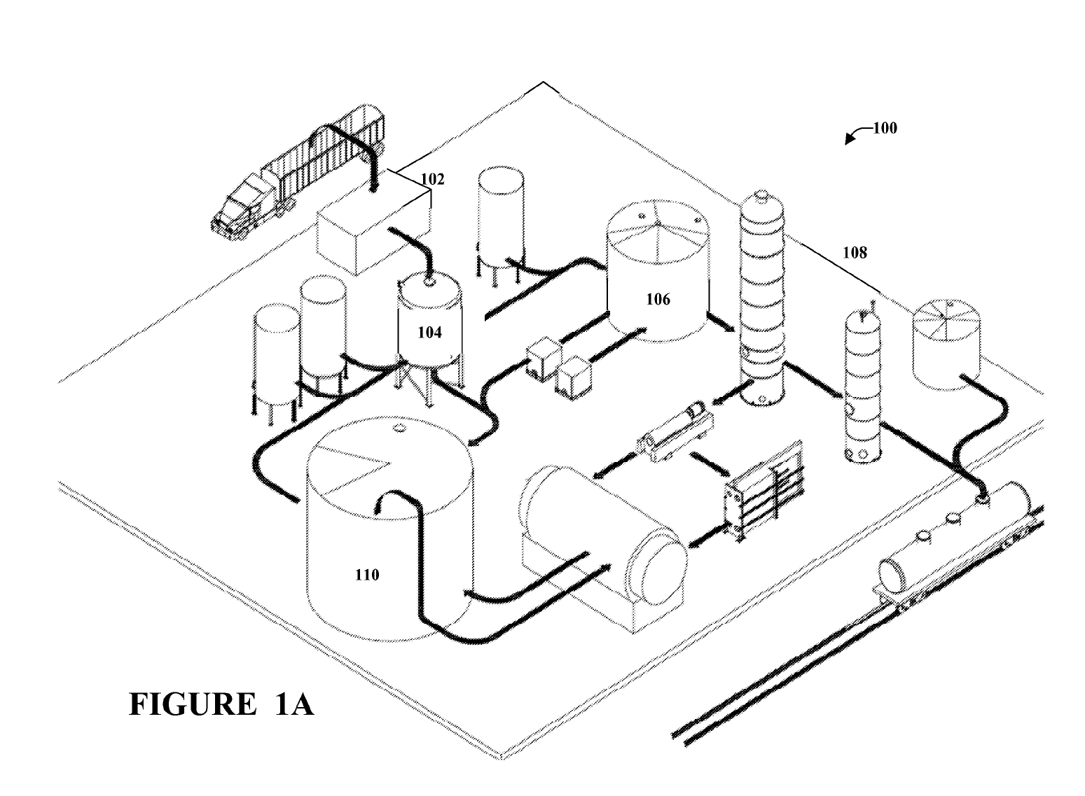

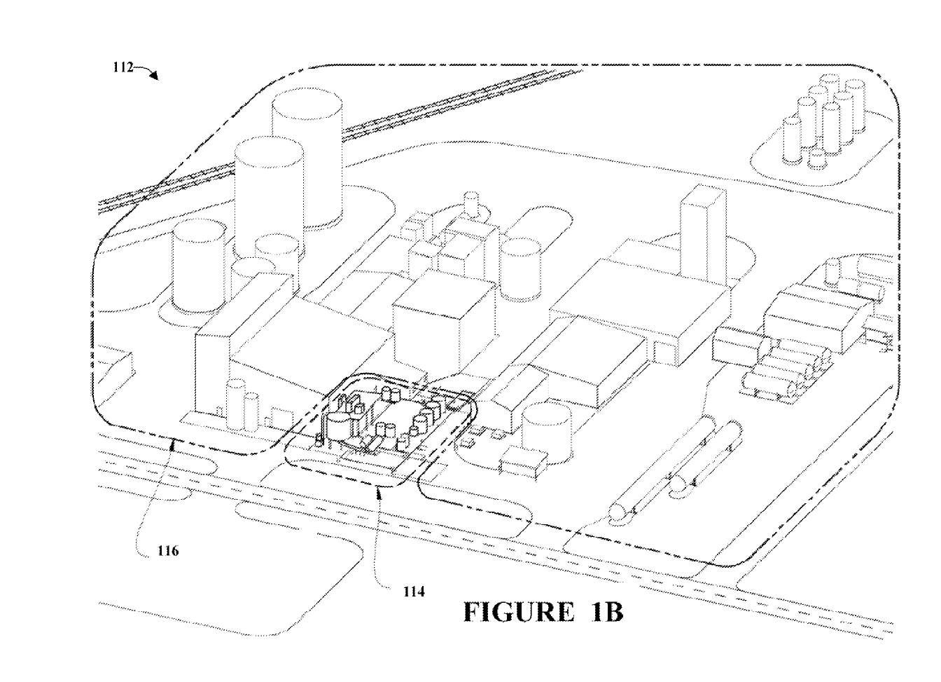

Referring to FIG. 1A, an example biorefinery 100 comprising an ethanol production facility configured to produce ethanol from biomass is shown. The example biorefinery 100 comprises an area where biomass is delivered and prepared to be supplied to the ethanol production facility. The cellulosic ethanol production facility comprises an apparatus for preparation 102, pre-treatment 104 and treatment of the biomass into treated biomass suitable for fermentation into fermentation product in a fermentation system 106. The cellulosic ethanol production facility comprises a distillation system 108 in which the fermentation product is distilled and dehydrated into ethanol. As shown in FIG. 1A, a waste treatment system 110 is shown as comprising an anaerobic digester and a generator. According to other alternative embodiments, the waste treatment system may comprise other equipment configured to treat, process, and recover components from the cellulosic ethanol production process, such as a solid/waste fuel boiler, anaerobic digester, aerobic digester or other biochemical or chemical reactors.

As shown in FIG. 1B, according to an exemplary embodiment, a biorefinery 112 may comprise a cellulosic ethanol production facility 114 (which produces ethanol from lignocellulosic material and components of the corn plant) co-located with a corn-based ethanol production facility 116 (which produces ethanol from starch contained in the endosperm component of the corn kernel). As indicated in FIG. 1B, by co-locating the two ethanol production facilities, certain plant systems may be shared, for example, systems for dehydration, storage, denaturing and transportation of ethanol, energy/fuel-to-energy generation systems, plant management and control systems, and other systems. Corn fiber (a component of the corn kernel), which can be made available when the corn kernel is prepared for milling (e.g. by fractionation) in the corn-based ethanol production facility, may be supplied to the cellulosic ethanol production facility as a feedstock. Fuel or energy sources, such as methane or lignin from the cellulosic ethanol production facility, may be used to supply power to either or both co-located facilities. According to other alternative embodiments, a biorefinery (e.g. a cellulosic ethanol production facility) may be co-located with other types of plants and facilities, for example an electric power plant, a waste treatment facility, a lumber mill, a paper plant, or a facility that processes agricultural products.

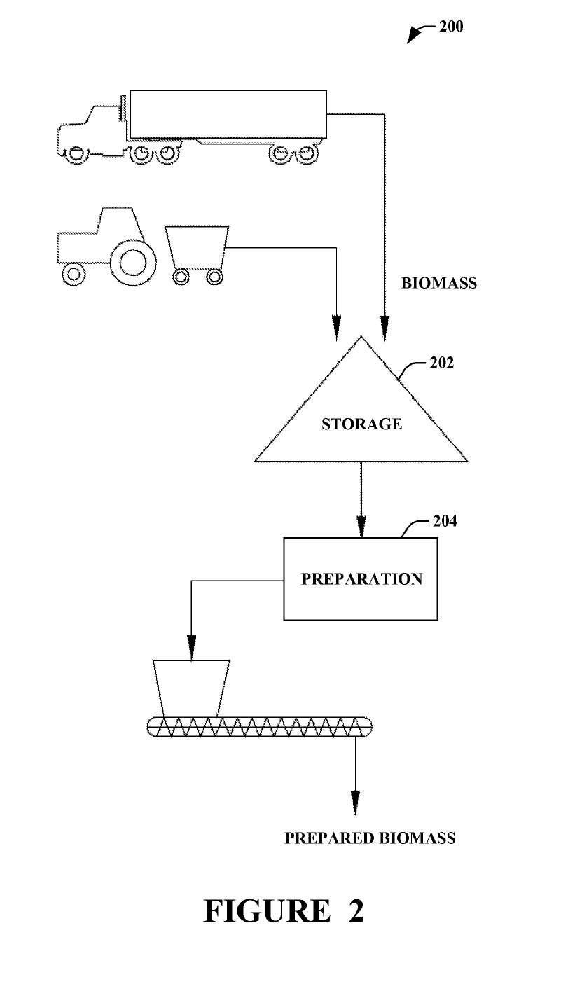

Referring to FIG. 2, a system 200 for preparation of biomass delivered to the biorefinery is shown. The biomass preparation system may comprise an apparatus for receipt/unloading of the biomass, cleaning (e.g. removal of foreign matter), grinding (e.g. milling, reduction or densification), and transport and conveyance for processing at the plant. According to an exemplary embodiment, biomass in the form of corn cobs and stover may be delivered to the biorefinery and stored 202 (e.g. in bales, piles or bins, etc.) and managed for use at the facility. According to an exemplary embodiment, the biomass may comprise at least about 20 to 30 percent corn cobs (by weight) with corn stover and other matter. According to other exemplary embodiments, the preparation system 204 of the biorefinery may be configured to prepare any of a wide variety of types of biomass (e.g. plant material) for treatment and processing into ethanol and other bioproducts at the plant.

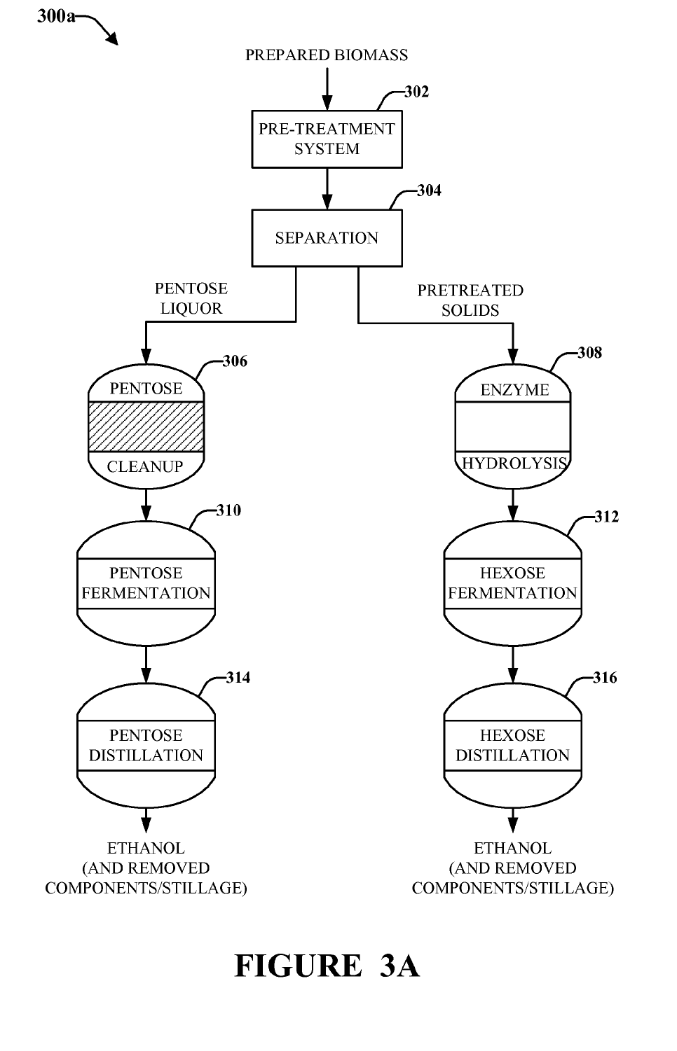

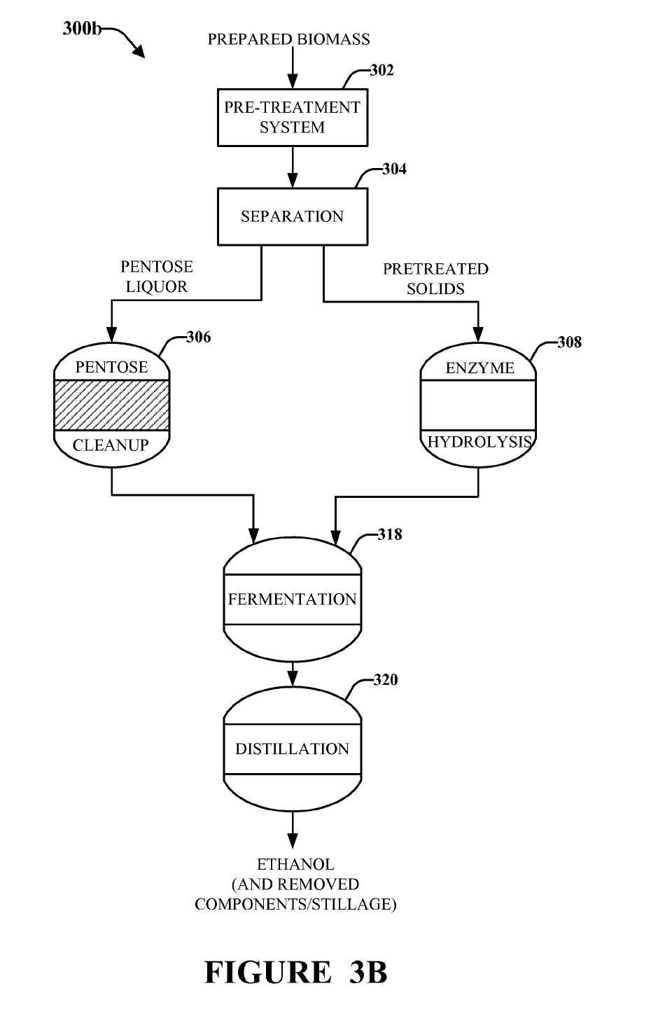

Referring to FIGS. 3A and 3B, alternate embodiments of a schematic diagram of the cellulosic ethanol production facility 300a and 300b are shown. According to some embodiments, biomass comprising plant material from the corn plant is prepared and cleaned at a preparation system. After preparation, the biomass is mixed with water into a slurry and is pre-treated at a pre-treatment system 302. In the pre-treatment system 302, the biomass is broken down (e.g. by hydrolysis) to facilitate separation 304 into a liquid component (e.g. a stream comprising the C5 sugars, known as pentose liquor) and a solids component (e.g. a stream comprising cellulose from which the C6 sugars can be made available). The C5-sugar-containing liquid component (C5 stream or pentose liquor) may be treated in a pentose cleanup treatment system 306. Further explanation of the pentose cleanup treatment system and methods will be discussed below in detail. In a similar manner, the C6-sugar-containing pretreated solids component may be treated in a solids treatment system using enzyme hydrolysis 308 to generate sugars. According to an embodiment, hydrolysis (such as enzyme hydrolysis) may be performed to access the C6 sugars in the cellulose; treatment may also be performed in an effort to remove lignin and other non-fermentable components in the C6 stream (or to remove components such as residual acid or acids that may be inhibitory to efficient fermentation).

In accordance with the embodiment of FIG. 3A, the treated pentose liquor may then be fermented in a pentose fermentation system 310, and the fermentation product may be supplied to a pentose distillation system 314 for ethanol recovery. In a similar manner, the treated solids, not including substantial amounts of C6 sugars, may be supplied to a hexose fermentation system 312, and the fermentation product may be supplied to a hexose distillation system 316 for ethanol recovery.

In the alternate embodiment of FIG. 3B, the resulting treated pentose liquor and treated solids may be combined after treatment (e.g. as a slurry) for co-fermentation in a fermentation system 318. Fermentation product from the fermentation system 318 is supplied to a combined distillation system 320 where the ethanol is recovered. According to any embodiment, a suitable fermenting organism (ethanologen) can be used in the fermentation system. In accordance with some aspects, the selection of an ethanologen may be based on various considerations, such as the predominant types of sugars present in the slurry. Dehydration and/or denaturing of the ethanol produced from the C5 stream and the C6 stream may be performed either separately or in combination.

During treatment of the C5 and/or C6 stream, components may be processed to recover byproducts, such as organic acids and lignin. The removed components during treatment and production of ethanol from the biomass from either or both the C5 stream and the C6 stream (or at distillation) can be treated or processed into bioproducts or into fuel (such as lignin for a solid fuel boiler or methane produced by treatment of residual/removed matter such as acids and lignin in an anaerobic digester) or recovered for use or reuse.

According to an embodiment, the biomass comprises plant material from the corn plant, such as corn cobs, corn plant husks and corn plant leaves and corn stalks (e.g. at least the upper half or three-quarters portion of the stalk). In accordance with some aspects, the composition of the plant material (e.g. cellulose, hemicellulose and lignin) will be approximately as indicated in TABLES 1A and 1B (e.g. after at least initial preparation of the biomass, including removal of any foreign matter). According to an embodiment, the plant material comprises corn cobs, husks/leaves and stalks; for example, the plant material may comprise (by weight) up to 100 percent cobs, up to 100 percent husks/leaves, approximately 50 percent cobs and approximately 50 percent husks/leaves, approximately 30 percent cobs and approximately 50 percent husks/leaves and approximately 20 percent stalks, or any of a wide variety of other combinations of cobs, husks/leaves and stalks from the corn plant. See TABLE 1A. According to an alternative embodiment, the lignocellulosic plant material may comprise fiber from the corn kernel (e.g. in some combination with other plant material). TABLE 1B provides various ranges believed to be representative of the composition of biomass comprising lignocellulosic material from the corn plant. According to exemplary embodiments, the lignocellulosic plant material of the biomass (from the corn plant) may comprise (by weight) cellulose at about 30 to 55 percent, hemicellulose at about 20 to 50 percent, and lignin at about 10 to 25 percent. According to another exemplary embodiment, the lignocellulosic plant material of the biomass (e.g. cobs, husks/leaves and stalk portions from the corn plant) may comprise (by weight) cellulose at about 35 to 45 percent, hemicellulose at about 24 to 42 percent, and lignin at about 12 to 20 percent. According to a particular embodiment, pre-treatment of the biomass may yield a liquid component that comprises (by weight) xylose at no less than 1.0 percent and a solids component that comprises (by weight) cellulose (from which glucose can be made available) at no less than 45 percent.

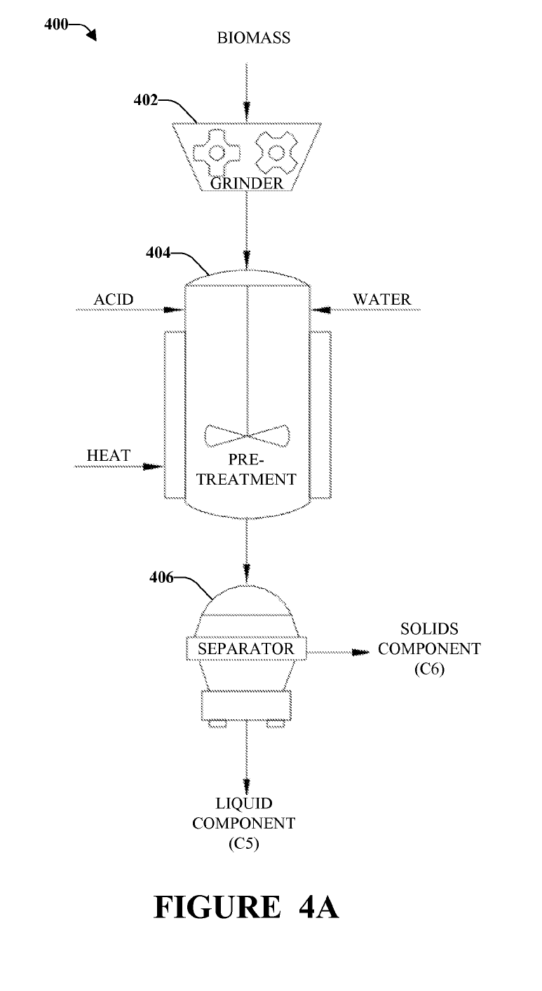

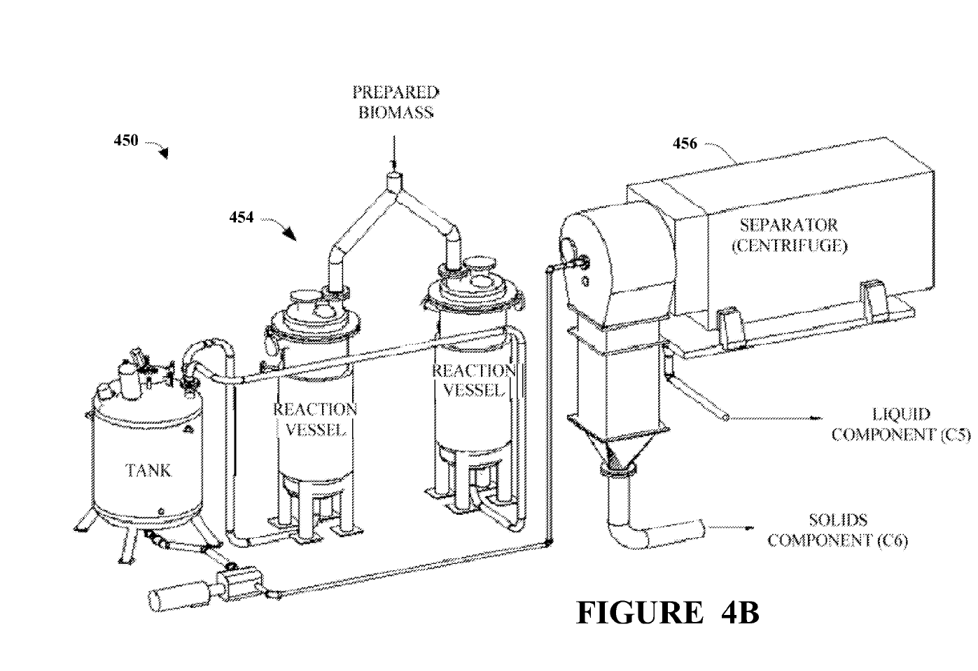

FIGS. 4A and 4B show exemplary apparatuses 400, 450 used for preparation, pre-treatment, and separation of lignocellulosic biomass according to an exemplary embodiment. As shown, biomass is prepared in a grinder 402 (e.g. a grinder or other suitable apparatus or mill). Pre-treatment of the prepared biomass is performed in a reaction vessel 404 (or set of reaction vessels 454) supplied with prepared biomass and acid/water in a predetermined concentration (or pH) and other operating conditions. The pre-treated biomass can be separated in a separator 406. As shown in FIG. 4B, the pre-treated biomass can be separated in a centrifuge 456 into a liquid component (C5 stream comprising primarily liquids with some solids) and a solids component (C6 stream comprising liquids and solids such as lignin and cellulose from which glucose can be made available by further treatment).

According to an embodiment, pre-treatment of biomass can be performed described in U.S. patent Ser. No. 12/716,984 entitled “SYSTEM FOR PRE-TREATMENT OF BIOMASS FOR THE PRODUCTION OF ETHANOL”, which is incorporated by reference in its entirety.

According to an embodiment, in the pre-treatment system an acid may be applied to the prepared biomass to facilitate the breakdown of the biomass for separation into the liquid (pentose liquor) component (C5 stream from which fermentable C5 sugars can be recovered) and the solids component (C6 stream from which fermentable C6 sugars can be accessed). According to some embodiments, the acid can be applied to the biomass in a reaction vessel under determined operating conditions (e.g. acid concentration, pH, temperature, time, pressure, solids loading, flow rate, supply of process water or steam, etc.) and the biomass can be agitated/mixed in the reaction vessel to facilitate the breakdown of the biomass. According to exemplary embodiments, an acid such as sulfuric acid, hydrochloric acid, nitric acid, phosphoric acid, acetic acid, etc. (or a formulation/mixture of acids) can be applied to the biomass. According to a particular embodiment, sulfuric acid may be applied to the biomass in pre-treatment. According to a particular embodiment, the prepared biomass may be pretreated with approximately 0.8 to 1.3 percent acid (such as sulfuric acid) and about 12 to 25 percent biomass solids at a temperature of approximately 130 to 180 degrees Celsius for approximately 5 to 12 minutes. The pre-treatment may also comprise a steam explosion step, where biomass is heated to and held at (e.g. hold time) approximately 155 to 160 degrees Celsius under pressure (e.g. 100 psi) at a pH of about 1.4 to 1.6, and the pressure is released to further aid in the breakdown of cellulose. After pretreatment the pre-treated biomass is separated into a solids component (C6) and a liquid pentose liquor component (C5), as shown in FIGS. 4A and 4B.

The liquid pentose liquor component (C5 stream) comprises water, dissolved sugars (such as xylose, arabinose and glucose) to be made available for fermentation into ethanol, acids and other soluble components recovered from the hemicellulose. (TABLE 2B provides typical and expected ranges believed to be representative of the composition of biomass comprising lignocellulosic material from the corn plant.) According to an exemplary embodiment, the liquid component may comprise approximately 5 to 7 percent solids (e.g. suspended/residual solids such as partially hydrolysed hemicellulose, cellulose, and lignin). According to a particular embodiment, the liquid component may comprise at least 2 to 4 percent xylose (by weight). According to other exemplary embodiments, the liquid component may comprise no less than 1 to 2 percent xylose (by weight). TABLES 2A and 2B list the composition of the liquid component of pre-treated biomass (from prepared biomass as indicated in TABLES 1A and 1B) according to exemplary and representative embodiments.

The solids component (C6 stream) comprises water, acids and solids such as cellulose from which sugar, such as glucose, can be made available for fermentation into ethanol, and lignin. (TABLE 3B provides ranges that can be representative of the composition of biomass comprising lignocellulosic material from the corn plant.) According to an exemplary embodiment, the solids component may comprise approximately 10 to 40 percent solids (by weight) (after separation). According to a particularly preferred embodiment, the solids component may comprise approximately 20 to 30 percent solids (by weight). According to another embodiment, the solids in the solids component may comprise no less than about 30 percent cellulose and the solids component may also comprise other dissolved sugars (e.g. glucose and xylose). TABLES 3A and 3B list the composition of the solids component of pre-treated biomass (from prepared biomass as indicated in TABLES 1A and 1B) according to exemplary and representative embodiments.

During pre-treatment, the severity of operating conditions (such as pH, temperature, and time) may cause formation of components that are inhibitory to fermentation. For example, under some conditions, the dehydration of sugars (such as xylose or arabinose) may cause the formation of furfural. Acetic acid may also be formed, for example, when acetate is released during the break down of hemicellulose in pre-treatment. The levels of acetic acid can become as high as 4000 ppm (0.4% w/v). Acetic acid is known to inhibit yeast metabolism. Also, acetic acid can inhibit xylose uptake and metabolism in the recombinant yeast. Reducing the acetic acid levels to about 2000 ppm or less has helped improve the fermentability of pentose liquor from corn cobs. Sulfuric acid, which may be added to prepared biomass to facilitate pre-treatment, if not removed or neutralized, may also be inhibitory to fermentation. According to an exemplary embodiment, by adjusting pre-treatment conditions (such as pH, temperature, and time), the formation of inhibitors can be reduced or managed; according to other exemplary embodiments, components of the pre-treated biomass may be given further treatment to remove or reduce the level of inhibitors (or other undesirable matter).

Treatment of the C5 stream (liquid component) of the biomass may be performed in an effort to remove components that are inhibitory to efficient fermentation (e.g. furfural, hydroxymethylfurfural (HMF), sulfuric acid and acetic acid) and residual lignin (or other matter) that may not be fermentable from the C5 sugar component so that the sugars (e.g. xylose, arabinose, as well as other sugars such as glucose) are available for fermentation. The C5 sugars in the C5 stream may also be concentrated to improve the efficiency of fermentation (e.g. to improve the titer of ethanol for distillation).

As noted above, fermentation inhibitors may traditionally be mitigated using ion exchange resins, over-liming, or large yeast inoculation of the fermentation step. There has been a substantial amount of research performed related to the use of over-liming as a way to reduce the effects of the fermentation inhibitors produced as a result of dilute acid pretreatment of lignocellulosic biomass. It has been concluded that the major drawbacks of the over-liming process are the loss in fermentable sugar (Pienkos and Zhang, 2009), sugar degradation due to hydroxide-catalyzed degradation reactions (Mohagheghi et al. 2006); and possible downstream effects in distillation. These downstream effects may include the precipitated calcium salts that may contaminate distillation columns, evaporators and heat exchangers, and the possibility of lactic acid bacterial contamination of the over-limed pentose liquor. This form of bacterial contamination may be particularly important since calcium lactate is inhibitory to the fermenting yeast (Pattison and vonHoly, 2001).

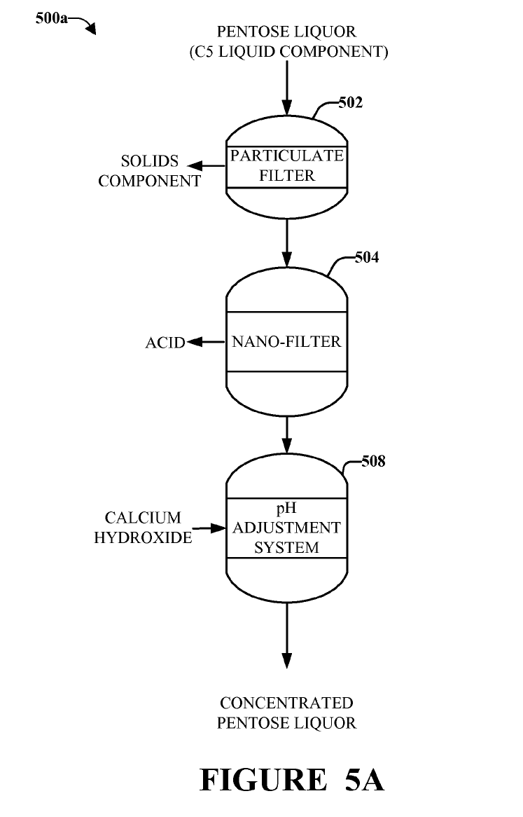

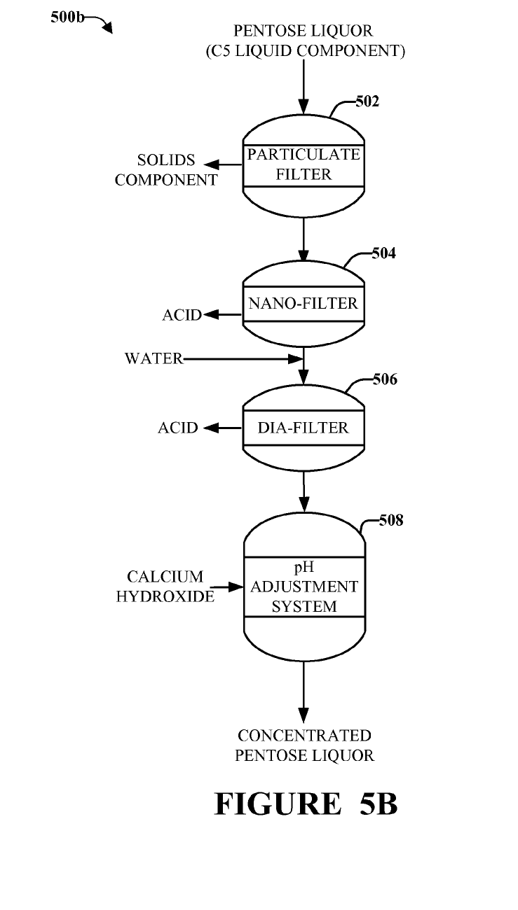

Provided herein are systems and methods for inhibitor mitigation using a combination of nano-filtration and addition of calcium hydroxide at reduced levels in order to increase fermentability of the pentose liquor without the common drawbacks of over-liming. FIG. 5A illustrates a first schematic perspective view of an inhibitor mitigation system 500a, in accordance with some embodiments. In this exemplary illustration, the pentose liquor (C5 liquid component) is provided to a series of filters known collectively as a filtration system. The filtration system may use one stage or multiple stages to treat the liquid component. In some embodiments, the filtration system may include a particulate filter 502 that removes particles and precipitates, which may interfere with the downstream nano-filters. In some embodiments, the particulate filter may have a pore size of approximately 0.1 to 20 micrometers to remove a solid component from the C5 stream. After removal of particulates, the pentose liquor may be passed through a nano-filter 504. The pentose liquor tends to include furfural, acetic acid, and other inhibitors to the downstream fermentation process. Treating the pentose liquor by nano-filtration membranes reduces the acetic acid levels, and possibly some of the other inhibitory compounds. Generally, the nano-filter 504 has a membrane configured with pores to allow water molecules and acid ions to pass through as permeate while retaining (larger molecular weight/size) sugar molecules as retentate.

FIG. 5B illustrates a similar system 500b wherein a second nano-filtration stage is present after the first nano-filter 504. The second nano-filter is a diafilter 506 configured for diafiltration in which additional water may be added to the liquid component to facilitate the flow (of water and acid) through the membrane (as permeate) and the retention of filtered and concentrated C5 sugars (as retentate).

After nanofiltration, the treated pentose liquor may be supplied to a pH adjustment tank 508 for adjustment of the pH of the liquor to roughly between 5.5 and 6.0. The pH adjustment is helpful for facilitation of fermentation; additionally the anti-inhibitor properties of the calcium hydroxide may be utilized to further clean the pentose liquor. However, unlike the over-liming procedures that are utilized to remove inhibitors, the volume of calcium hydroxide utilized in the disclosed embodiments is substantially reduced, thereby avoiding the numerous drawbacks associated with over-liming methods.

After pH adjustment, the clean pentose liquor may be supplied to an evaporator to evaporate excess liquids, thereby increasing the xylose concentration, in some embodiments. This stage may be optional since excess water may already have been removed during nanofiltration. The resulting concentrated pentose liquor is now ready for fermentation into ethanol.

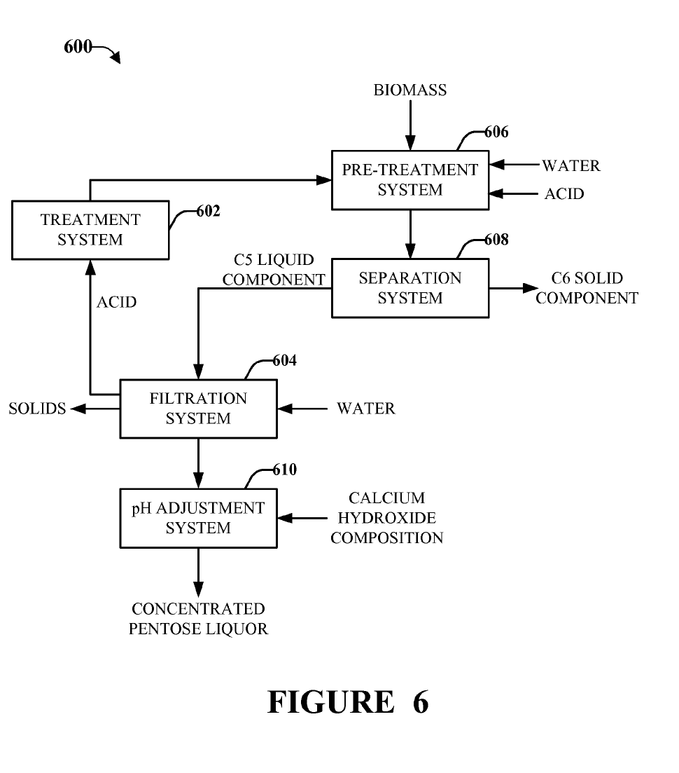

FIG. 6 provides an example of a process flow 600 where the acid is treated at a treatment system 602 and re-used. According to an exemplary embodiment, acid that has been removed from the liquid component by a filtration system 604 can be recovered and supplied for re-use in a pre-treatment system 606. The pretreatment system 606 may break down incoming biomass using acid, mechanical, and enzymatic processes as discussed above. The pretreated biomass may be separated into the liquid and solid components at a separation system 608. The liquid component may be supplied to the filtration system 604 for acid removal. According to an embodiment, at least about 60 to 80 percent of acetic acid and at least about 40 to 50 percent of sulfuric acid can be removed from the liquid component in treatment with the nano-filtration system following acid pre-treatment (e.g. using dilute sulfuric acid) and separation of the biomass. The acid can be further treated at the treatment system 602 to concentrate the acid to a desired concentration (e.g. 2 percent). The concentration of the removed acid can be performed for example by removing water by reverse osmosis (RO).

According to a particular embodiment, the filtration system 604 may comprise a filter with a pore size of less than 10 nm. The filter may be operated under approximately 150 to 600psi pressure to achieve a suitable feed rate. An example of a suitable filter is the Dow Filmtec NF4040, available from Dow Chemical Company in Midland, Mich.

Filtered liquid component may then be supplied to the pH adjustment system 610 for adjustment of the liquor's pH to about 5.5 to 6.0. Adjustment of pH may comprise the inclusion of at least some lime (Ca(OH)₂). After pH adjustment, clean concentrated pentose liquor is generated, which may be supplied for fermentation into ethanol.

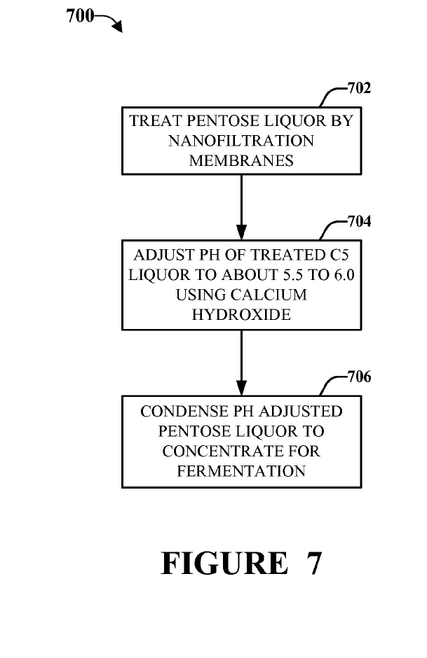

FIG. 7 is a process flow diagram of the inhibitor mitigation system, in accordance with some embodiments. The flow process 700 begins with the treatment (at 702) of the pentose liquor (C5 liquid component) by nano-filtration. As noted previously, nano-filtration may remove substantial amounts of various inhibitory compounds including acetic acid, etc. The pentose liquor, in some embodiments, may be filtered for particulates prior to nano-filtration to avoid fouling of the membranes. The nanofilter treated pentose liquor may then be pH adjusted (at 704) using calcium hydroxide (lime) alone, or in combination with some other base (e.g. potassium hydroxide or ammonium hydroxide). This may further reduce inhibitory compounds found in the pentose liquor.

After pH adjustment of the pentose liquor, the clean pentose liquor may optionally be subjected to concentration (at 706) utilizing reverse osmosis or an evaporator. In some embodiments, the nanofiltration may sufficiently concentrate the liquor as to eliminate the need for further concentration. For example, the treatment system shown as a filtration system in FIG. 5B can be used to concentrate the sugars in the liquid component (C5 stream) by at least 1.5 to 2.25 fold.

The concentrated, nanofiltered pentose liquor may then be supplied to a fermentation system alone, or as a slurry with degraded C6 components, in order to generate ethanol and other byproducts.

Alternatively, after nano-filtration, the pentose liquor may first be concentrated and subsequently pH adjusted in the fermentation vessel using calcium hydroxide, in some embodiments. By pH adjusting after the evaporation step, the risk of calcium buildup in the evaporator may be minimized.

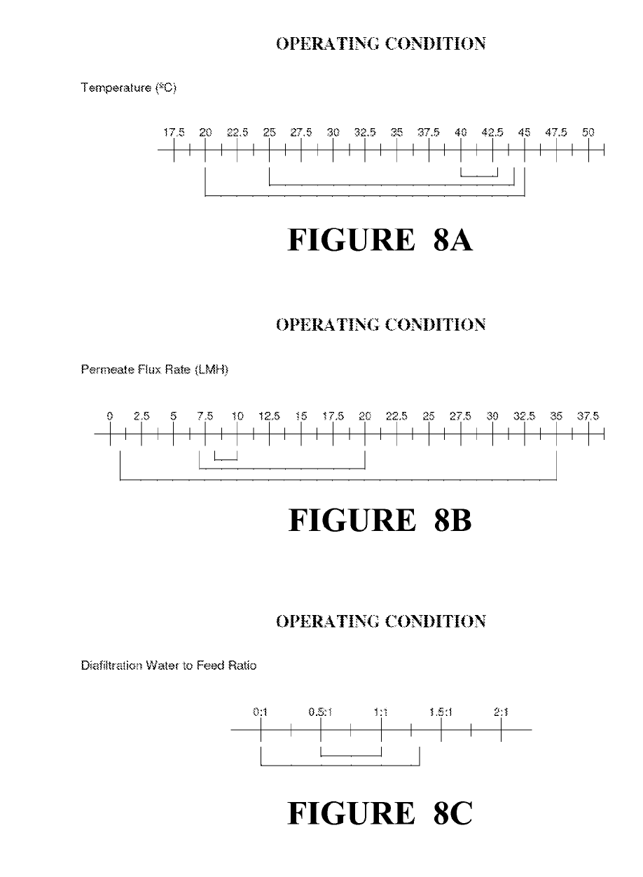

Exemplary operating conditions relating to the filtration system are shown in FIGS. 8A through 8C. Operating conditions for each subject condition can be indicated as “nested” ranges, comprising an acceptable operating range (the outer/wide range shown), a second operating range (the middle range shown, if applicable), and a particular operating range (the inner/narrow range shown, if applicable). As shown in FIG. 8A, a typical temperature range for operating the filter is from 20 to 45 degrees Celsius. In another embodiment, the temperature range is 25 to 44 degrees Celsius. In a particular embodiment, the temperature range is 40 to 43 degrees Celsius.

As shown in FIG. 8B, a typical permeate flux rate for the first nano-filtration step is 1.5 to 35 L/m²/h (or LMH). In another embodiment, the flux rate is 7 to 20 LMH. In a particular embodiment, the flux rate is 8 to 10 LMH. As shown in FIG. 8C, a typical ratio of added water to liquid component feed for diafiltration is 0 to 1.3; and in another embodiment the ratio is 0.5 to 1.1.

A series of limited examples were conducted according to an exemplary embodiment of the system (as shown in FIG. 5B) in an effort to determine suitable apparatus and operating conditions for the treatment of lignocellulosic hydrolysate to improve fermentation. The following examples are intended to provide clarity to some embodiments and means of operation; given the limited nature of these examples, it does not limit the scope of the disclosed aspects.

### EXAMPLE 1

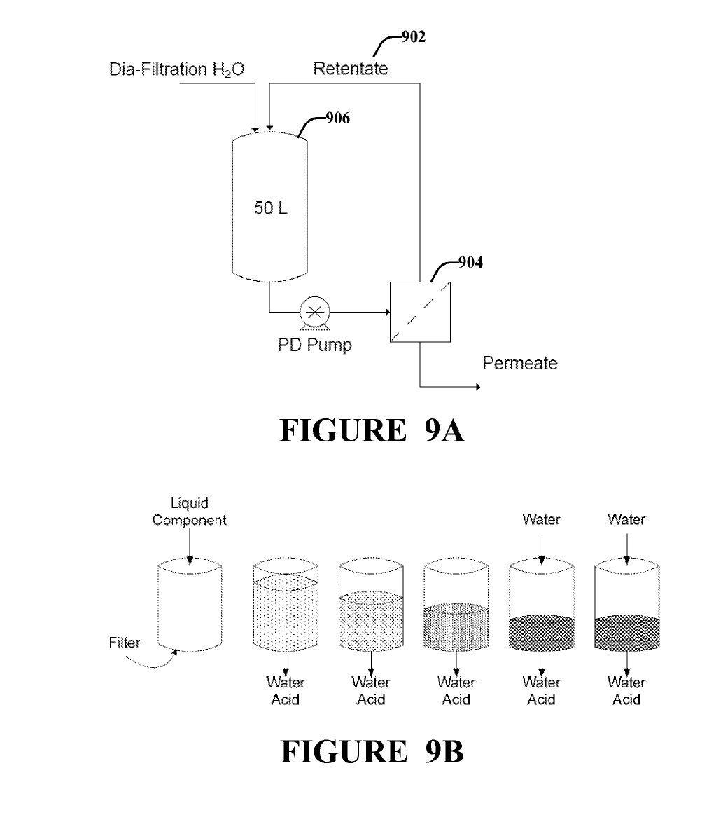

Acid removal from the liquid component was tested according to an experimental design shown in TABLE 4A, using an experimental process shown in FIG. 9A. Three different filters were tested: Dow Filmtec NF-4040, Dow Filmtec NF-270 (both available from Dow Chemical Company, Midland Mich.), and Koch SeIRO MPS-34 (available from Koch Membrane Systems, Inc., Wilmington, Mass.). All three filters were spiral-wound membrane filters with 4-inch diameter and 40-inch length. The filters were operated at 25 degrees Celsius, and the Dow Filmtec NF-270 was operated at 32 degrees Celsius. The multistage nano-filtration system was modeled by the experimental process shown in FIG. 9A, where retentate 902 from the filter 904 can be cycled back into the storage/feed tank 906 and filtered again to simulate a second or consecutive stage. The principle of concentration and diafiltration is illustrated in FIG. 9B.

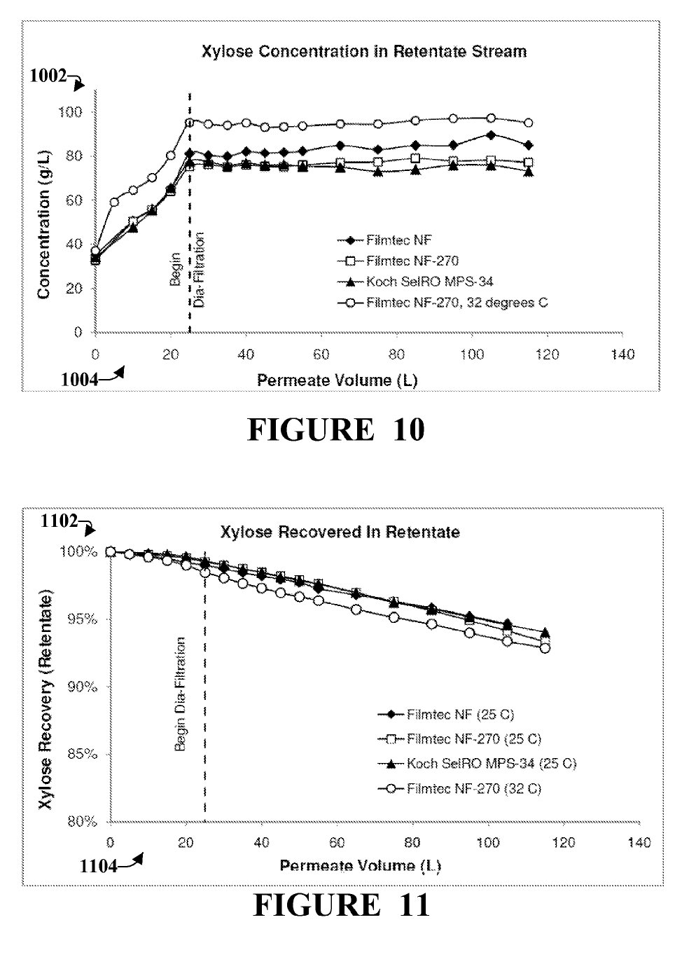

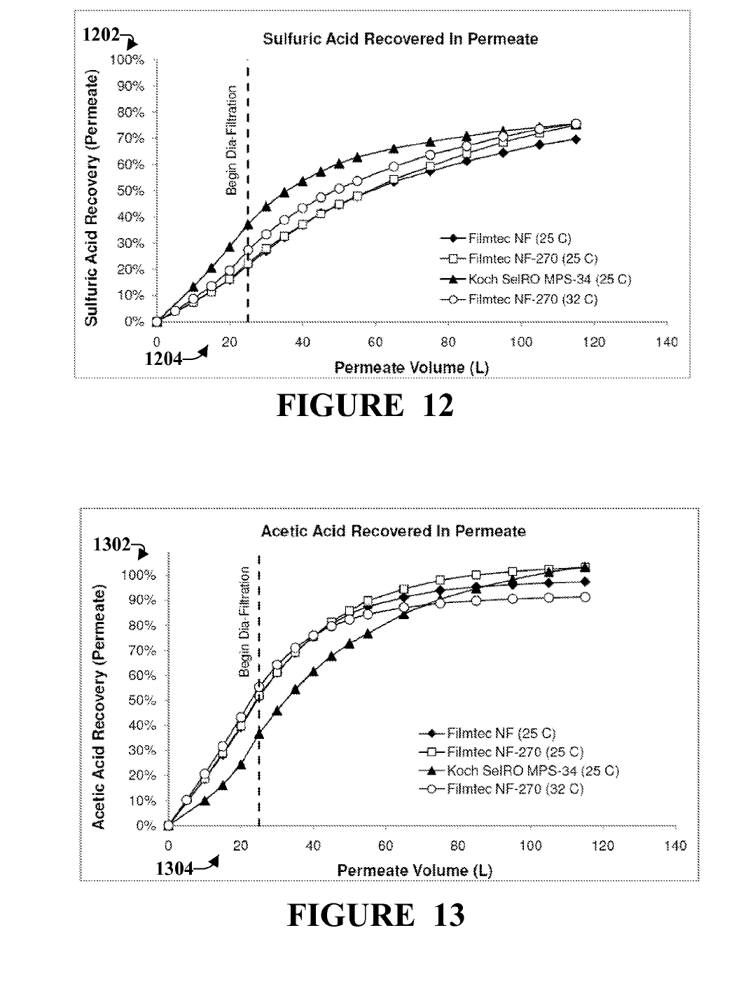

The liquid component was pre-filtered using a 10 micrometer filter. The vessel was filled with 45 L of pre-treated biomass liquid component, and approximately 1 mL of an anti-foaming agent (KFO-119, available from Kabo Chemicals, Inc., Cheyenne, Wy.) was added to prevent foaming. The liquid component was concentrated until approximately 25 L of permeate had passed through the membrane filter, and approximately 20 L of retentate remained, yielding an estimated 2.25× concentration of sugars in the retentate. The diafiltration stage was begun by adding water to the retentate in 5 L and 10 L increments according to the experimental design (TABLE 4A). For each incremental water addition, the equivalent amount of permeate was collected causing the retentate volume to remain constant. Samples of retentate and permeate streams were collected for analysis, and the results are shown in TABLE 4B and FIGS. 10 through 13. TABLE 4B shows the concentration of sulfuric acid, acetic acid, and xylose in the liquid component retentate before and after filtration. The start of diafiltration (e.g., addition of water) is indicated in the figures when the permeate volume reached 25 L. FIG. 10 shows xylose concentration in the retentate (at 1002) plotted versus permeate volume (at 1004). It was observed that prior to the start of diafiltration the xylose concentration increases sharply, and during diafiltration the xylose concentration remains relatively constant. FIG. 11 shows xylose recovery (at 1102) as a percentage versus the retentate volume (at 1104). FIG. 12 shows sulfuric acid (at 1202) recovery in the permeate (at 1204). FIG. 13 shows acetic acid recovery (at 1302) in the permeate (at 1304).

It was also observed that when permeate volume reached 45 L (equal to the initial volume of liquid component sample), 97 percent or more of the xylose remained in the retentate, and over 41 percent of the sulfuric acid and over 67 percent of the acetic acid was removed into the permeate. It was further observed that the Filmtec NF-270 filter was most effective in removing acetic acid with 81.3 percent of acetic acid and 41.2 percent of sulfuric acid removed and a 98.2 percent retention of xylose. The Koch SelRO filter was most effective for removing sulfuric acid with 57.4 percent of sulfuric acid and 67.8 percent of acetic acid removed and a 98.1 percent retention of xylose.

### EXAMPLE 2

Acid removal from the liquid component was tested according to an experimental design shown in TABLE 5A, using an experimental process shown in FIG. 9A. The experiment was conducted using a Dow Filmtec NF filter (available from Dow Chemical Company, Midland Mich.). The Dow Filmtec NF filter is a spiral-wound membrane filter with 4-inch diameter and 40-inch length. The filter was operated at ambient temperature (approximately 22 degrees Celsius). The multi-stage nano-filtration system was modeled by the experimental process shown in FIG. 9A, where permeate from the filter can be cycled back into the storage/feed tank and filtered again to simulate a second or consecutive stage.

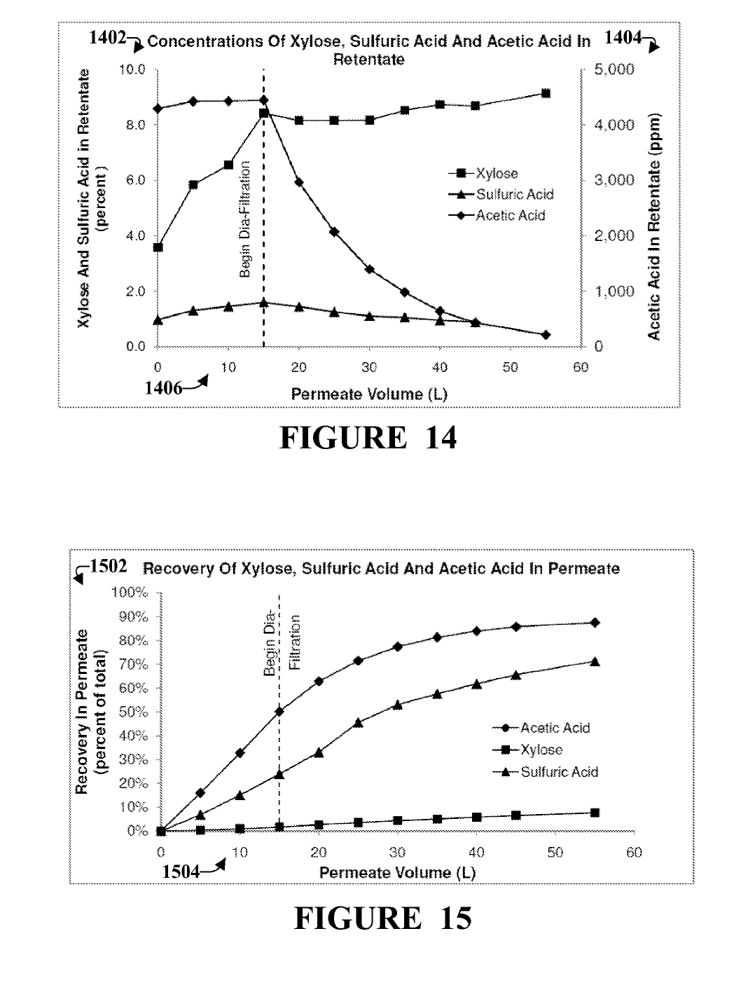

The liquid component was pre-filtered using a 1 micrometer filter. The vessel was filled with 30 L of pre-treated biomass liquid component and approximately 1 mL of an anti-foaming agent (KFO-119, available from Kabo Chemicals, Inc., Cheyenne, Wy.) was added to prevent foaming. The liquid component was concentrated until approximately 15 L of permeate had passed through the membrane filter and approximately 15 L of retentate remained, yielding an estimated 2× concentration of sugars in the retentate. The diafiltration stage was begun by adding water to the retentate in 5 L and 10 L increments according to the experimental design (TABLE 5A). For each incremental water addition, the equivalent amount of permeate was collected causing the retentate volume to remain constant. Samples of retentate and permeate streams were collected for analysis; the results are shown in TABLE 5B and FIGS. 14 and 15. FIG. 14 shows xylose concentration, sulfuric acid concentration (at 1402) and acetic acid concentration (at 1404) in the retentate as a function of the permeate volume (at 1406). FIG. 15 shows xylose recovery, sulfuric acid recovery and acetic acid recovery as a percentage in the permeate (at 1502) versus permeate volume (at 1504). It was observed that when permeate volume reached 30 L (equal to the initial volume of liquid component sample), about 96 percent of the xylose remained in the retentate, and about 53 percent sulfuric acid and about 77 percent of acetic acid was removed to the permeate.

### EXAMPLE 3

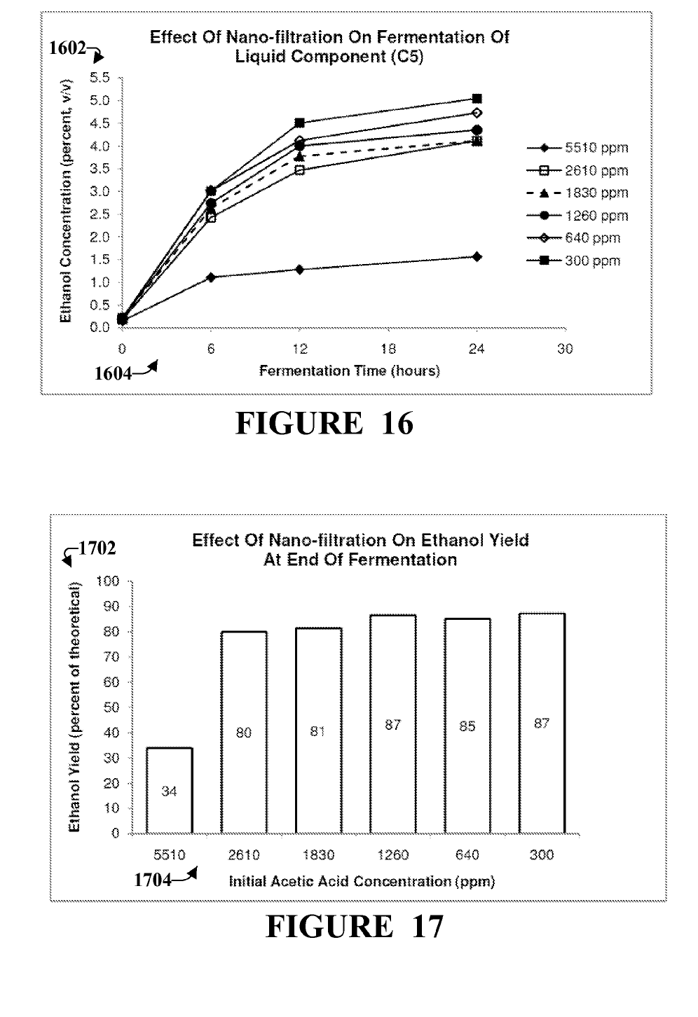

Samples of retentate from Example 2 were collected during diafiltration and were fermented to test the effect of treatment on fermentation efficiency. Samples with different levels of acetic acid were collected. The samples were fermented using 10 g/L (dry weight) of a genetically modified strain of Saccharomyces cerevisiae yeast (as described in U.S. Pat. No. 7,622,284, assigned to Royal Nedalco B.V.). Each fermentor was supplied with 5 mg/L of Lactoside (available from Lallemand Ethanol Technology, Milwaukee, Wis.), 62.5 g/L urea and 1 g/L yeast extract, and the pH was adjusted to 5.5 using KOH. The fermentations were conducted at 32 degrees Celsius. The fermentors were sampled and tested for xylose and ethanol concentration. The results for 24 hours of fermentation are shown in FIG. 16 where ethanol concentration (at 1602) is plotted versus fermentation time (at 1604). Similarly, FIG. 17 illustrates the ethanol yields (at 1702) at the completion of fermentation versus initial acetic acid concentrations (at 1704). The sample with an initial acetic acid level of 5510 ppm took longer to finish, and reached an ethanol concentration of 0.8 percent and a yield of 34 percent (of theoretical maximum) by 48 hours. It was observed that the samples with lower acetic acid levels performed better. It was also observed that when the initial acetic acid level was 5510 ppm, only 30 percent of the sugar was converted to ethanol by 24 hours, but when the initial acetic acid level was between 1830 and 2610 ppm, a yield of at least 80 percent could be achieved. It was further observed that when the initial acetic acid level was 1260 or less, a yield of at least 85 percent could be achieved.

### EXAMPLE 4

In the fourth example, three samples of pentose liquor were treated via nano-filtration, as described above, and then pH adjusted using potassium hydroxide or calcium hydroxide to a pH value of about 6.0. The studies were conducted in 125 mL Erlenmeyer flasks with 60 mL final volume of the pentose liquor. The pH of the liquors were adjusted to 6.0 prior to inoculation of yeast. Yeast extract and urea were added at 1 g/L and 0.06 g/L, respectively as nutrients. Antibacterial agent, lactoside 247, was added at 5 ppm final concentration. The yeast strain RN1016 cultured in shake flasks in Yeast extract, Peptone (YP) media with glucose (1%) and xylose (2%) was added to the various liquors at 0.5 g/L. The flasks were placed in a water bath shaker at 32° C. (shaking at 125 rpm). Samples were withdrawn periodically and analyzed for sugars, organic acids and ethanol using high performance liquid chromatography (HPLC).

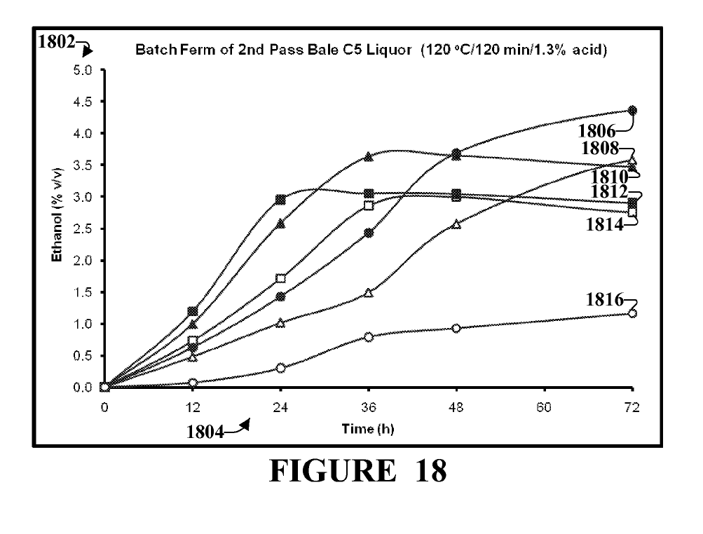

At FIG. 18, the resulting ethanol concentrations (at 1802) are shown plotted against fermentation time (at 1804) for each sample. The initial xylose concentrations were 5% w/v (squares); 6% w/v (triangles) and 7.5% w/v (circles). Samples were pH adjusted using potassium hydroxide (open symbols) or using calcium hydroxide (filled symbols). At the 72 hour fermentation mark, 7.5% w/v xylose liquor adjusted with calcium hydroxide 1806 provided the greatest ethanol yield, followed by 6% w/v xylose liquor adjusted with potassium hydroxide 1808, followed by 6% w/v xylose liquor adjusted with calcium hydroxide 1810, followed by 5% w/v xylose liquor adjusted with calcium hydroxide 1812, followed by 5% w/v xylose liquor adjusted with potassium hydroxide 1814, and lastly 7.5% w/v xylose liquor adjusted with potassium hydroxide 1816.

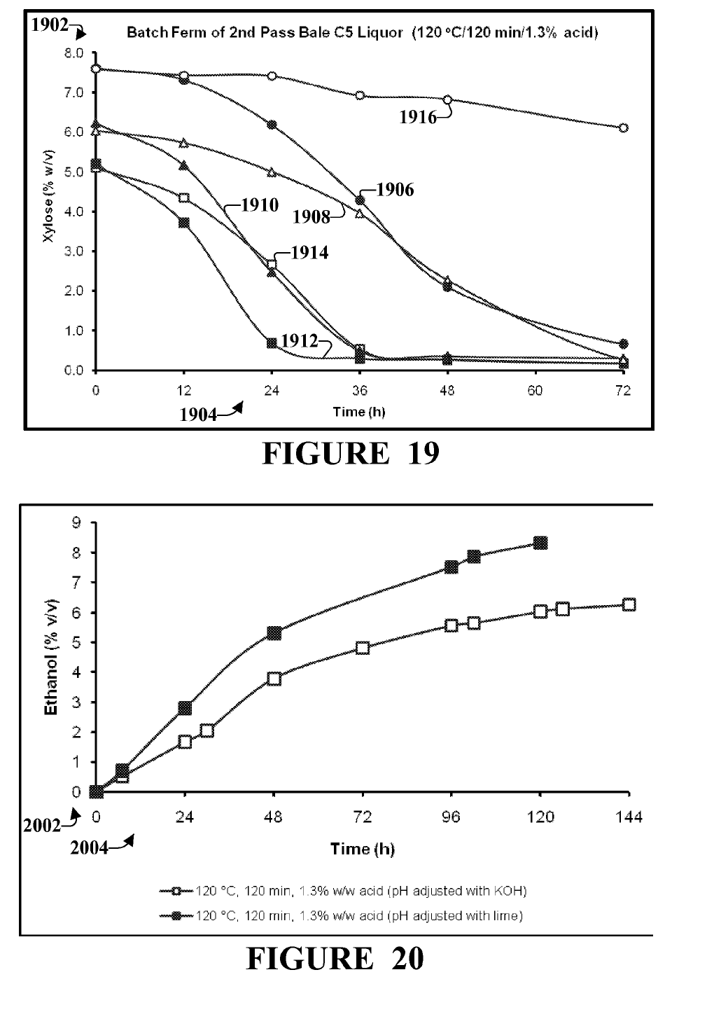

In a similar manner, at FIG. 19 the xylose concentrations 1902 of the samples are shown plotted against fermentation time 1904. Samples are labeled 1906 for 7.5% w/v xylose liquor adjusted with calcium hydroxide, 1908 for 6% w/v xylose liquor adjusted with potassium hydroxide, 1910 for 6% w/v xylose liquor adjusted with calcium hydroxide, 1912 for 5% w/v xylose liquor adjusted with calcium hydroxide, 1914 for 5% w/v xylose liquor adjusted with potassium hydroxide, and lastly 1916 for 7.5% w/v xylose liquor adjusted with potassium hydroxide.

The results show that using calcium hydroxide in place of potassium hydroxide for pH adjustment makes the nF (nano-filtration) treated pentose liquor more fermentable especially at the higher initial xylose concentration tested. The fermentation efficiencies were also better when lime was used for pH adjustment. An efficiency of about 78% was observed when the initial xylose concentration was 7.5% w/v. Whereas the observed fermentation efficiency was only roughly 25% when potassium hydroxide was used for pH adjustment of the liquor with 7.5% w/v xylose. Even at the lower initial xylose concentrations tested, faster rates of fermentation were observed in reactors pH adjusted with lime compared to the reactors pH adjusted with potassium hydroxide.

### EXAMPLE 5

In the fifth example, pentose liquor from acid steeping of second pass bale material at 120° C. for 2 hours and 1.3% acid was used. This pentose liquor was subjected to nano-filtration (nF). This nano-filtration treated liquor was evaporated to concentrate the xylose further. This concentrated, nano-filtration treated liquor was used for feeding the fermentor (Fed-batch process).

Clarified thin stillage was added at 1 g/L. Similar to the experiments where lime was used for pH adjustment, instead of pumping the liquor continuously after 24 hours of batch fermentation, the liquor was added (fed) in batches at three different time points. The feed was performed in such a way that the concentration of xylose at the end would be the same as in fermentations that were continuously fed with xylose liquor. The experiments with lime had to be modified due to the observed foaming and some precipitation of solids which made it very difficult for continuous feeding of the liquor at a constant rate. For all the fed-batch fermentations, the antibacterial agent lactoside 247 was added at 5 ppm. Urea was added at 0.24 g/L. The yeast strain RN1016 was aerobically propagated using the developed standardized protocol and added. The yeast loading to the yeast propagator was at 0.5 g/L. The fed-batch fermentations were maintained at 32° C. for the entire length. The pH of the fermentations were not controlled; however, the fermentations were set at pH of 5.5 or 6.0 using potassium hydroxide or lime at the outset. At 24 hours of fermentation, the pH was readjusted up to 5.5 with the respective base in each study, however, the pH was not continuously maintained throughout the fermentation. Samples were withdrawn at various intervals and analyzed for sugars, organic acids and ethanol using HPLC.

Results from the fermentations are illustrated in FIG. 20, where the ethanol concentration (at 2002) is plotted against fermentation time (at 2004). The results indicate that the use of lime (calcium hydroxide) for pH adjustment of the nano-filtration treated pentose liquor from acid steeping of second pass bales improves the fermentability of the liquor.

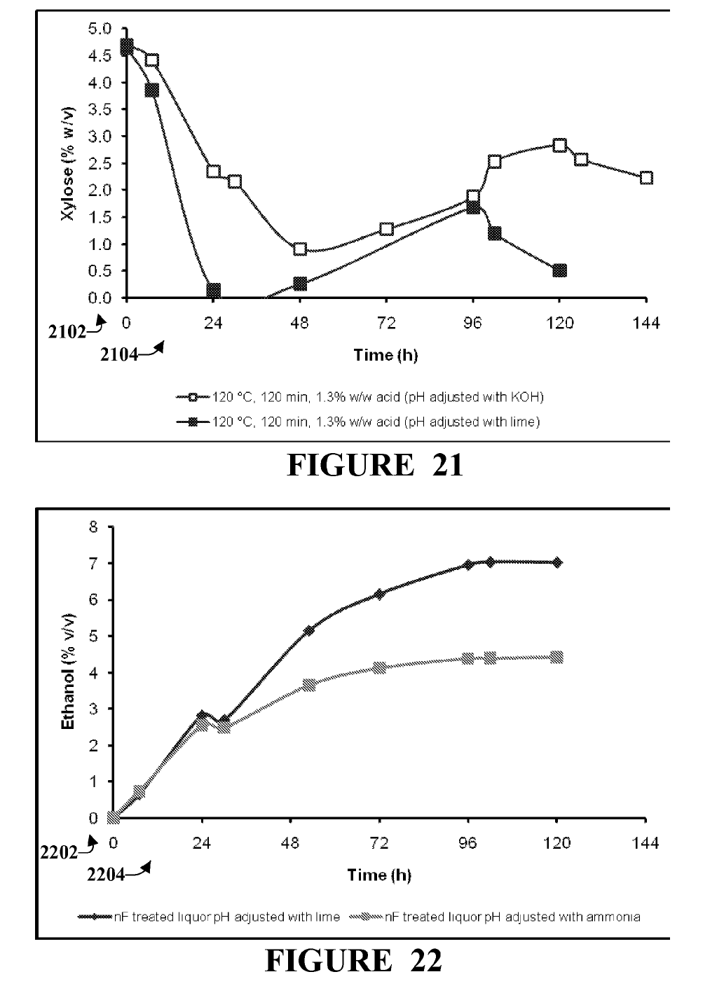

In a similar manner, at FIG. 21 the concentration of xylose sugar (at 2102) is seen plotted versus the fermentation time (at 2104). Using lime for pH adjustment of nano-filtration treated pentose liquor increased the efficiency of fermentation (sugar to ethanol conversion) from roughly 61% with potasium hydroxide (KOH) to 84% for lime (Ca(OH)₂). The most likely reason for this may be the binding of some of the inhibitors (lignin degradation compounds) by the calcium hydroxide.

### EXAMPLE 6

In the sixth example, since potassium hydroxide is relatively expensive, and ammonium hydroxide is already commonly used in ethanol production facilities for pH adjustment, an attempt was made to study the use of ammonium hydroxide as a base for pH adjustment. Moreover, the use of ammonium hydroxide provides a source of nitrogen for the fermenting organism and there will be no concerns of lime related scaling issues in distillation columns and/or heat exchangers.

Samples were prepared and processed as described above in relation with example 5, however, instead of pH adjustment with potassium hydroxide for one sample, ammonium hydroxide was utilized.

The results from the study are summarized in FIG. 22 where the percent ethanol (at 2202) is plotted versus fermentation time (at 2204). Again, lime was a better base than ammonium hydroxide for pH adjustment in the fed-batch fermentation, especially when trying to feed higher sugar concentrations to achieve higher ethanol titers. However, ammonium hydroxide use in the yeast aerobic propagation gave a good cell yield (˜10 g/L) in the 17-hour mark.

Further, the residual xylose after 120 hours in the reactor that was pH adjusted with ammonium hydroxide was 4.4% w/v. Whereas, in the reactor that was pH adjusted with lime, the residual xylose level was only 0.35% w/v. These results suggest that ammonium hydroxide likely does not bind the inhibitors in the C5 liquor as well as lime.

### EXAMPLE 7

In the seventh example, since there might be downstream concerns when lime is used (e.g., scaling in distillation columns and evaporators, and membrane fouling), an attempt was made at reducing the overall lime usage. The approach consists of using ammonium hydroxide for pH adjustment during yeast propagation, and a combination of lime and ammonium hydroxide for initial pH adjustment. In this example, calcium hydroxide was used to adjust the pH of the nano-filtration treated pentose liquor up to 4.0, followed by use of ammonium hydroxide to adjust the pH up to 5.5. This was compared against using only lime (Ca(OH)₂) to adjust to a pH of 5.5 in the fed-batch fermentation. The aerobic yeast propagation on pentose liquor from second pass bales was performed using the standard procedure with an inoculum size of 0.5 g/L produced over 10 g/L in 17 hours with ammonium hydroxide used for pH adjustment. This yeast was used to inoculate the fermentations. In the fed-batch fermentations, the urea dosages used were 0.24 g/L (4 mM) when lime was used for pH adjustment and only 0.06 g/L (1 mM) when lime and ammonium hydroxide were used for pH adjustment.

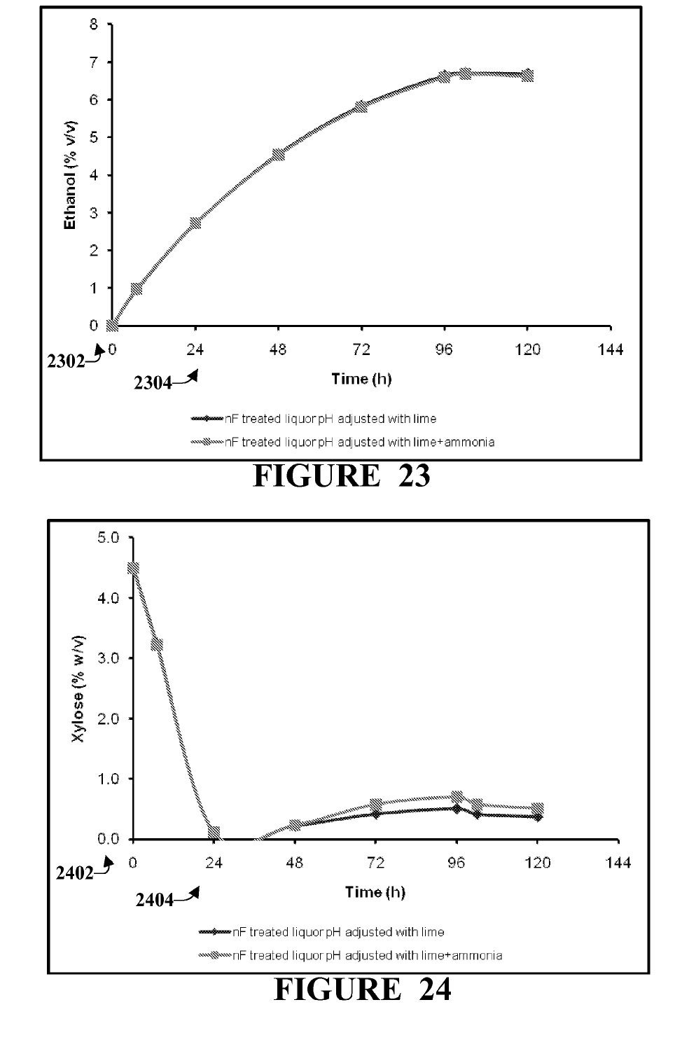

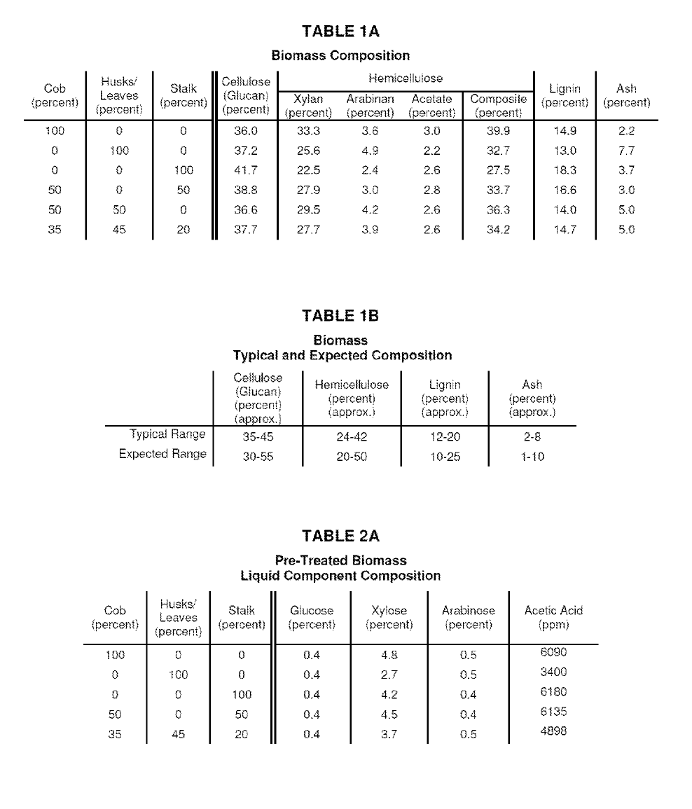

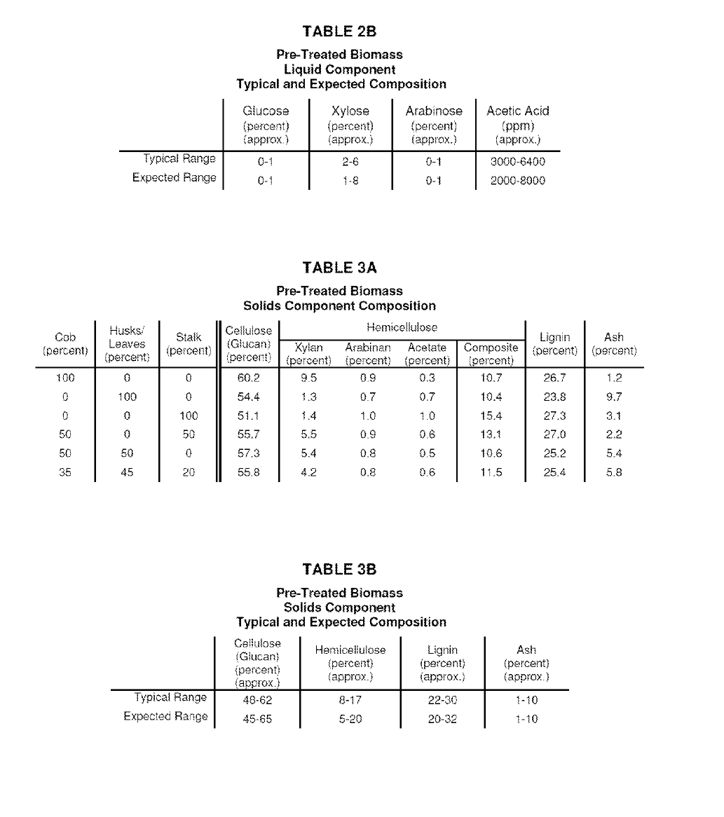

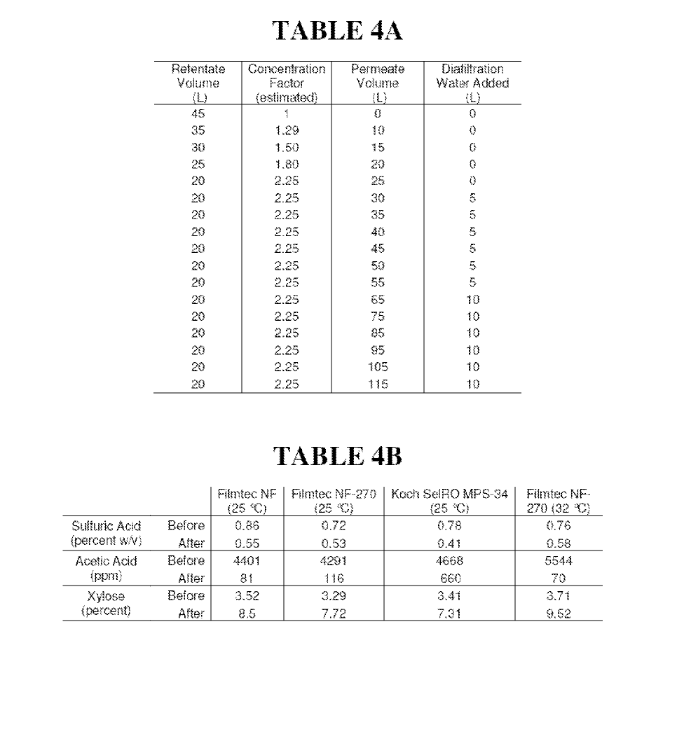

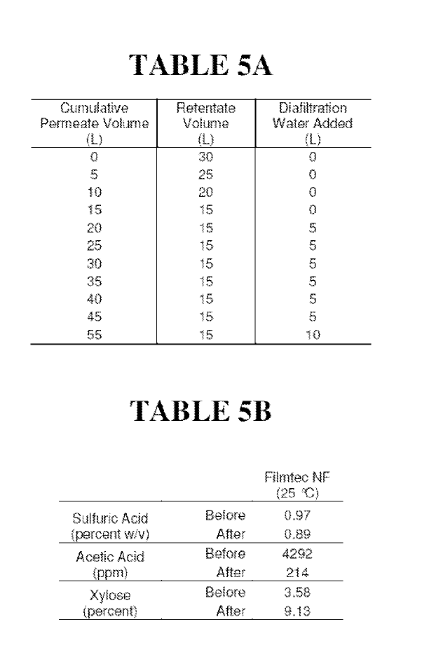

Referencing FIG. 23, the ethanol concentrations (at 2302) are plotted versus fermentation time (at 2304). At FIG. 24, residual xylose concentrations (at 2402) are plotted versus fermentation time (at 2404). No major differences were observed in the ethanol titers obtained with respect to both the approaches tested. In both the fermentations, about 6.8% v/v ethanol was obtained in 96 to 100 hours of fermentation. This corresponds to an efficiency of about 79%. Using ammonium hydroxide in combination with lime helps reduce lime usage. Additionally, the pH dropped to about 4.7 at the end of fermentation. Further reducing the pH in the beer to less than 3.8 using sulfuric acid may reduce the calcium oxalate formation during distillation.

The embodiments as disclosed and described in the application (including the FIGURES and Examples) are intended to be illustrative and explanatory of the present invention. Modifications and variations of the disclosed embodiments, for example, of the apparatus and processes employed (or to be employed) as well as of the compositions and treatments used (or to be used), are possible; all such modifications and variations are intended to be within the scope of the disclosed aspects.

The word “exemplary” is used to mean serving as an example, instance, or illustration. Any embodiment or design described as “exemplary” is not necessarily to be construed as preferred or advantageous over other embodiments or designs, nor is it meant to preclude equivalent exemplary structures and techniques known to those of ordinary skill in the art. Rather, use of the word exemplary is intended to present concepts in a concrete fashion, and the disclosed subject matter is not limited by such examples.

The term “or” is intended to mean an inclusive “or” rather than an exclusive “or.” To the extent that the terms “comprises,” “has,” “contains,” and other similar words are used in either the detailed description or the claims, for the avoidance of doubt, such terms are intended to be inclusive in a manner similar to the term “comprising” as an open transition word without precluding any additional or other elements.

What is claimed is:

1. A system for treating a liquid component separated from biomass to yield a treated liquid component comprising sugars available to be fermented into a fermentation product comprising:
a filter configured to remove matter having a particle size of larger than about 25 microns from the liquid component;
at least one nanofilter configured to remove acids and concentrate xylose in the filtered liquid component; and
an apparatus configured to adjust a pH of the nanofiltered liquid component using a calcium hydroxide composition.

2. The system of claim 1, wherein the biomass comprises lignocellulosic material, wherein the lignocellulosic material comprises at least one of corn cob, corn plant husk, corn plant leaves, and corn plant stalks.

3. The system of claim 1, wherein the filter has a pore size of 0.1 to 20 micrometers.

4. The system of claim 1, wherein the at least one nanofilter includes a first nanofiltration stage and a second nanofiltration stage.

5. The system of claim 4, wherein the second nanofiltration stage comprises a membrane with pores that allow water molecules and acid ions to pass as permeate and retain sugar molecules as retentate, wherein the nanofiltered liquid component comprises the retentate.

6. The system of claim 4, wherein the second nanofiltration stage is configured for diafiltration, wherein the diafiltration comprises water added to the liquid component in a ratio of 0:1 to 1.3:1.

7. The system of claim 4, wherein the first nanofiltration stage has a permeate flux rate of 1.5 to 35 L/m²/h.

8. The system of claim 1, wherein the apparatus adjusts the pH of the nanofiltered liquid component to about 5.5 to 6.0 using calcium hydroxide.

9. The system of claim 1, wherein the apparatus adjusts the pH of the nanofiltered liquid component to about 5.5 to 6.0 using a combination of calcium hydroxide and at least one of ammonium hydroxide and potassium hydroxide.

10. The system of claim 9, wherein the apparatus adjusts the pH of the nanofiltered liquid component to about 4.0 using calcium hydroxide and then adjusts the pH to about 5.5 to 6.0 with at least one of ammonium hydroxide and potassium hydroxide.

11. A method for treating a liquid component separated from biomass to yield a treated liquid component comprising sugars available to be fermented into a fermentation product comprising:
removing matter having a particle size of larger than about 25 microns from the liquid component;
removing acids and concentrate xylose in the liquid component; and
adjusting a pH of the liquid component using a calcium hydroxide composition.

12. The method of claim 11, wherein the biomass comprises lignocellulosic material that comprises at least one of corn cob, corn plant husk, corn plant leaves, and corn plant stalks.

13. The method of claim 11, wherein removing the matter comprises using a filter with a pore size of 0.1 to 20micrometers.

14. The method of claim 11, wherein the removing comprises using at least one nanofilter including a first nanofiltration stage and a second nanofiltration stage.

15. The method of claim 14, wherein the second nanofiltration stage comprises a membrane with pores that allow water molecules and acid ions to pass as permeate and retain sugar molecules as retentate, wherein the liquid component comprises the retentate.

16. The method of claim 14, wherein the second nanofiltration stage is configured for diafiltration, wherein the diafiltration comprises adding water to the liquid component in a ratio of 0:1 to 1.3:1.

17. The method of claim 14, wherein the first nanofiltration stage has a permeate flux rate of 1.5 to 35 L/m²/h.

18. The method of claim 11, wherein the adjusting the pH of the liquid component comprises adjusting the pH to about 5.5 to 6.0 using calcium hydroxide.

19. The method of claim 11, wherein the adjusting the pH of the liquid component comprises adjusting the pH to about 5.5 to 6.0 using a combination of calcium hydroxide and at least one of ammonium hydroxide and potassium hydroxide.

20. The method of claim 19, wherein the adjusting the pH of the liquid component comprises adjusting the pH to about 4.0 using calcium hydroxide and then adjusting the pH to about5.5 to 6.0 with at least one of ammonium hydroxide and potassium hydroxide.
